(19) United States
(12) Patent Application Publication
(10) Pub. No.: US 2016/0022659 A1
(43) Pub. Date: Jan. 28, 2016

(54) METHODS FOR TREATING ATTENTION DEFICIT HYPERACTIVITY DISORDER USING A COMBINATION OF BUPROPION ((±)-2-(TERT-BUTYLAMINO)-1-(3-CHLOROPHENYL)PROPAN-1-ONE) AND PHENTERMINE (2-METHYL-1-PHENYLPROPAN-2-AMINE)

(71) Applicant: Thomas R. Winston, Leawood, KS (US)

(72) Inventor: Thomas R. Winston, Leawood, KS (US)

(21) Appl. No.: 14/337,666

(22) Filed: Jul. 22, 2014

(51) Int. Cl. A61K 31/4458; A61K 45/06; A61K 31/137

(52) U.S. Cl. CPC A61K 31/4458; A61K 31/137; A61K 45/06

(57) ABSTRACT

Compositions and methods for treating attention deficit hyperactivity disorder are disclosed. Compositions for treating attention deficit hyperactivity disorder include bupropion, phentermine and a psychostimulant drug. Methods for treating attention deficit hyperactivity disorder include administering to an individual in need a composition including bupropion and phentermine Methods for treating attention deficit hyperactivity disorder can also include further administering to an individual in need a psychostimulant drug.

### BACKGROUND OF THE DISCLOSURE

The present disclosure is generally directed to methods and compositions for treating attention deficit hyperactivity disorder. Methods for treating attention deficit hyperactivity disorder include administering to an individual in need a composition including a combination of bupropion and phentermine Compositions for treating attention deficit hyperactivity disorder include a combination of bupropion, phentermine and a psychostimulant drug.

Attention deficit hyperactivity disorder (ADHD) is a psychiatric disorder characterized by problems of attention, hyperactivity, disruptive behavior and impulsive acts. Lack of focus in school-aged individuals can lead to poor school performance ADHD can also contribute to problems with relationships. About half of the individuals diagnosed with ADHD during childhood continue to have symptoms into adulthood. Diagnosis of ADHD involves the appearance of symptoms between the age of six to twelve and their presence for more than six months.

Treatment of ADHD can involve the use of stimulant medications, counseling and lifestyle changes. The use of stimulant treatments, especially their use in children, is controversial because of the uncertainty of their long-term effects, possible side-effects and abuse potential. Accordingly, there is a need for alternative methods and compositions for treating ADHD.

### SUMMARY

The present disclosure is generally directed to methods for treating attention deficit hyperactivity disorder. More specifically, in one aspect, the present disclosure is directed to a method for treating attention deficit hyperactivity disorder in an individual in need comprising administering to the individual a composition that includes a combination of bupropion and phentermine.

In another aspect, the present disclosure is directed to composition comprising bupropion, phentermine and a psychostimulant drug.

### DETAILED DESCRIPTION

While the disclosure is susceptible to various modifications and alternative forms, specific embodiments thereof are herein described below in detail. It should be understood, however, that the description of specific embodiments is not intended to limit the disclosure to cover all modifications, equivalents and alternatives falling within the spirit and scope of the disclosure as defined by the appended claims.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, suitable methods and materials are described below.

### Methods for Treating ADHD

In one aspect, the present disclosure is directed to a method for treating ADHD in an individual in need. The method includes administering to an individual in need composition including a combination of bupropion and phentermine.

Bupropion as a single agent is effective for treating ADHD at a dose of 400 mg per day. Without being bound by theory, bupropion (formula I; (±)-2-(tert-Butylamino)-1-(3-chlorophenyl)propan-1-one) is an antidepressant that functions as a mild dopamine reuptake inhibitor and weak norepinephrine reuptake inhibitor. Bupropion also functions as a nicotinic acetylcholine receptor antagonist.

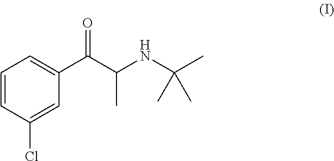 [SMILES: CC(NC(C)(C)C)C(=O)c1cccc(Cl)c1]

Bupropion has been reported for use as a treatment for ADHD in minors and adults, but results were inconclusive in a double-blind study of children. Evidence for treating ADHD with bupropion was weaker than for FDA-approved treatments.

Phentermine (formula II; 2-methyl-1-phenylpropan-2-amine) is generally recommended as an appetite-suppressant for short-term treatment of obesity because the development of tolerance to the compound over time. The U.S. Food and Drug Administration recommend that phentermine be used for up to 12 weeks. As an appetite-suppressant, the U.S. Food and Drug Administration also recommends a dosage of a half of a single 37.5 mg phentermine hydrochloride tablet up to a single 37.5 mg phentermine hydrochloride tablet (equivalent to 30 mg phentermine base) for patients with a body mass index of greater than 30 kg/m² (or greater than 27 kg/m² with other risk factors such as controlled hypertension, diabetes, and hyperlipidemia).

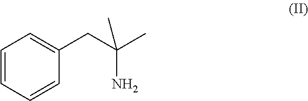

Without being bound by theory, phentermine acts on the hypothalamus portion of the brain to stimulate the adrenal glands to release norepinephrine. Phentermine can also stimulate the release of epinephrine or adrenaline.

In contrast to the administration of bupropion as a single agent for treating ADHD, the composition of the present disclosure including a combination of bupropion and phentermine allows for lower initial dosages of bupropion and/or phentermine than their dosages when administered as single agents. The lower initial dosages can contribute to a reduction in acquired tolerance. The lower dosages can also contribute to a reduction in the likelihood of side-effects caused by higher dosages. Additionally, the composition of the present disclosure allows for the administration of bupropion and/or phentermine over a prolonged period of time with less risk of dependence. Over time, and/or if tolerance develops, the dosage of bupropion and/or the dosage of phentermine can be increased for the continued treatment of ADHD. Thus, the lower initial dosages of bupropion and phentermine in the composition of the present disclosure can allow for a longer treatment period than when bupropion and/or phentermine are administered as single agents.

In another embodiment, the method can further include administration of a psychostimulant drug. As used herein, “psychostimulant drug” refers to psychoactive drugs that induce improvements in mental and/or physical functions including, for example, attention, alertness, wakefulness, productivity, heart rate, blood pressure, mood and locomotion by enhancing the activity of the central and peripheral nervous systems. Suitable psychostimulant drugs can be, for example, amphetamines, methylphenidate, dextroamphetamine ((2S)-1-phenylpropan-2-amine, d-alpha-methylphenethylamine dexamphetamine, dexamfetamine and DEXEDRINE®), ephedrine ((R*,S*)-2-(methylamino)-1-phenylpropan-1-ol), pseudoephedrine ((S,S)-2-methylamino-1-phenylpropan-1-ol), amphetamine ((RS)-1-phenylpropan-2-amine and (RS)-1-phenyl-2-aminopropane), methamphetamine (N-methyl-1-phenylpropan-2-amine), methylphenidate (methyl phenyl(piperidin-2-yl)acetate, CONCERTA® and RITALIN®), lisdexamfetamine ((2S)-2,6-diamino-N-[(2S)-1-phenylpropan-2-yl]hexanamide and VYVANSE®), cathinone ((S)-2-amino-1-phenyl-1-propanone), cathine ((1S,2 S)-2-amino-1-phenylpropan-1-ol), methcathinone ((RS)-2-(methylamino)-1-phenyl-propan-1-one), benzylpiperazine (1-benzylpiperazine), methylenedioxypyrovalerone ((RS)-1-(Benzo[d][1,3]dioxol-5-yl)-2-(pyrrolidin-1-yl)pentan-1-one), 4-methylaminorex (4-Methyl-5-phenyl-2-amino-oxazoline), pemoline ((RS)-2-amino-5-phenyl-1,3-oxazol-4(5H)-one and CYLERT®), phenmetrazine (3-methyl-2-phenylmorpholine and PRELUDIN®), phentermine, propylhexedrine ((RS)-N,α-dimethyl-cyclohexylethylamine and BENZEDREX® and OBESIN®) and combinations thereof.

Amphetamines stimulate the central nervous system and are used as single agents to treat ADHD and narcolepsy. Amphetamine exists as the two enantiomers, levoamphetamine (formula III; (RS)-1-phenylpropan-2-amine) and dextroamphetamine (formula IV; (RS)-1-phenyl-2-aminopropane)).

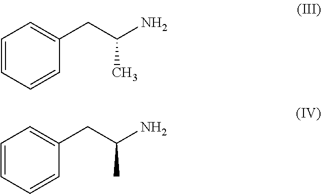

Suitable amphetamines can be, for example, amphetamine mixed salts (e.g., ADDERALL®), dextroamphetamine, levoamphetamine and lisdexamfetamine. The amphetamine mixed salts can include, for example, amphetamine salt and dextroamphetamine salt.

Methylphenidate (formula V; methyl phenyl(piperidin-2-yl)acetate) is a psychostimulant drug approved by the FDA for the treatment of ADHD. Administration of methylphenidate as a single agent for the treatment of ADHD can vary from about 10 mg to about 60 mg per day. Methylphenidate can result in increasing or maintaining alertness, combating fatigue and improving attention. Methylphenidate inhibits dopamine reuptake and also inhibits the reuptake of norepinephrine.

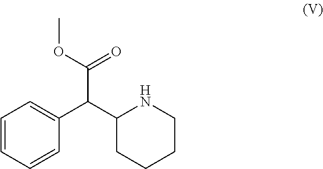 [SMILES: COC(=O)C(c1ccccc1)C1CCCCN1]

In yet another embodiment, the method for treating ADHD in an individual in need can further include administering atomoxetine ((3R)-N-methyl-3-(2-methylphenoxy)-3-phenylpropan-1-amine; (R)-N-methyl-3-phenyl-3-(o-tolyloxy)propan-1-amine; STRATTERA®). Atomoxetine is approved by the FDA for the treatment of ADHD and selectively inhibits norepinephrine reuptake.

In one embodiment, the method includes administering a composition including a therapeutically effective amount of bupropion, a therapeutically effective amount of phentermine and a therapeutically effective amount of a psychostimulant drug. As used herein, “therapeutically effective amount” refers to that amount that provides a therapeutic effect for a given condition and administration regimen. It can be understood, however, that the total daily usage of the compounds and compositions of the disclosure can be decided by the attending physician within the scope of sound medical judgment. The specific therapeutically effective dose level for any particular patient can depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. Actual dosage levels of active ingredients in the pharmaceutical compositions disclosed herein can be varied so as to obtain an amount of the active compound(s) that is effective to achieve the desired therapeutic response for a particular individual, composition and mode of administration. The selected dosage level can depend upon the activity of the particular compound, the route of administration, the severity of the condition being treated and the condition and prior medical history of the individual being treated. However, it is within the skill of the art to start doses of the compound at levels lower than required to achieve the desired therapeutic effect and to gradually increase the dosage until the desired effect is achieved. Upon achieving the desired therapeutic effect, a fixed ratio of each agent can be determined to form a concentrated dosage of the combination therapy to accommodate a volume of fluid such that the combination therapy can be administered over a period of time such as, for example, a 24 hour period.

The active compounds (e.g., bupropion and phentermine, and psychostimulant drugs) can be administered in a convenient manner such as by oral administration, injection (subcutaneous, intravenous, etc.), inhalation, transdermal application, or rectal administration. Depending on the route of administration, the active compound may be coated in a material to protect the compound from the action of enzymes, acids and other natural conditions which may inactivate the compound. A preferred route of administration is by oral administration.

As used herein, “dosage” refers to physically discrete units suited as unitary dosages for the mammalian individual to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

In using the combination therapy of the present disclosure, the same dose that is usually given as a single agent may be given through a normal administration route. In a particularly preferred embodiment, a reduced dose (for example, 0.10-0.99 times the highest dose as a single agent) can be given through a normal administration route.

A particularly suitable composition for oral administration can be a pill-form (for example, a tablet and a capsule) containing bupropion and phentermine. The dosage of bupropion can be, for example, from about 75 mg to about 400 mg, including from about 75 mg to about 100 mg, from about 75 mg to about 150 mg, from about 75 mg to about 200 mg, and from about 75 mg to about 300 mg. The dosage of phentermine can be, for example, from about 8 mg to about 37.5 mg, including from about 8 mg to about 10 mg, from about 8 mg to about 15 mg, and from about 8 mg to about 30 mg. In one particularly suitable embodiment, the composition can include about 400 mg of bupropion and from about 8 mg phentermine to about 37.5 mg phentermine. In another particularly suitable embodiment, the composition can include about 400 mg of bupropion and about 8 mg phentermine. In another particularly suitable embodiment, the composition can include about 400 mg of bupropion and about 10 mg phentermine. In another particularly suitable embodiment, the composition can include about 400 mg of bupropion and about 15 mg phentermine. In another particularly suitable embodiment, the composition can include about 400 mg of bupropion and about 30 mg phentermine. In another particularly suitable embodiment, the composition can include about 400 mg of bupropion and about 37.5 mg phentermine. For adults, the dosages of methylphenidate (RITALIN®) can be from about 10 mg/day to about 60 mg/day and can be administered in two to three divided doses per day. For adolescents age 13 to 17 years, the dosages of methylphenidate (CONCERTA®) can be from about 18 mg/day to about 72 mg/day. For adults 18 years and older, the dosages of methylphenidate (CONCERTA®) can be from about 18 mg/day to about 72 mg/day. For adults, the dosage of atomoxetine can be from about 20 mg/day to about 100 mg/day and can be administered in 2 divided doses per day.

For children age 6 years and older, the dosage of bupropion can be from about 1 mg/kg of body weight per day to about 6 mg/kg of body weight per day. For children age 6 years and older with a body weight up to 70 kg, the dosages of atomoxetine can be from about 0.5 mg/kg of body weight per day to about 1.4 mg/kg of body weight per day. For children age 6 years and older with a body weight over 70 kg, the dosages of atomoxetine can be from about 20 mg/day to about 40 mg/day. For children age 6 years and older, the dosages of methylphenidate (RITALIN®) can be from about 0.3 mg/kg of body weight per day to about 2 mg/kg of body weight per day in three (3) divided doses or from about 5 mg twice per day to about 30 mg twice per day up to 60 mg per day. For children age 6 to 12 years, the dosages of methylphenidate (CONCERTA®) can be from about 18 mg/day to about 54 mg/day.

The individual in need can be diagnosed with ADHD and/or suspected of having ADHD. The individual diagnosed with ADHD and/or suspected of having ADHD can be diagnosed with any of ADHD Predominantly Inattentive type, ADHD Predominantly Hyperactive-Impulsive type and ADHD Combined type. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of individuals “in need” of assistance in addressing one or more specific conditions noted herein), not all individuals will fall within the subset or subclass of individuals as described herein.

As used herein, the term “individual” refers to a male human or a female human. The individual can be, for example, a human. The individual can be an adult individual, an adolescent child (age 6 to 18 years) and a child (less than 6 years).

The effect of administering the composition including a combination of bupropion and phentermine, and further with a psychostimulant drug, can be confirmed by an improvement in the individual's attention, restlessness, behavior and/or impulsivity in comparison with that of the individual's attention, restlessness, behavior and/or impulsivity prior to administration as can be determined and/or observed by one skilled in the art such as, for example, medical professionals, teachers, parents and combinations thereof.

The compositions of the present disclosure can be administered in any manner effective for delivering the compositions. Exemplary modes of administration may be, for example, orally, topically, transdermally, parenterally, enterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. Particularly suitable administration can be orally.

The compounds described herein are administered as a combination. It will be appreciated that when using the composition, the compounds may be in the same pharmaceutically acceptable carrier at a specific ratio that is administered simultaneously in the same pharmaceutically acceptable carrier to achieve the desired effect. Buproprion and its metabolites such as, for example, R,R-hydroxybupropion, S,S-hydroxybupropion, threo-hydrobupropion and erythro-hydrobupropion, can be monitored for example, by measurement in the blood and using positron emission tomography of the brain. Similarly, phentermine and its metabolites can be measured in the blood. When included, psychostimulant drugs and their metabolites can be measured in the blood. The ratio of each compound can be determined and/or adjusted by one skilled in the art using methods known by those skilled in the art such as, for example, measuring buproprion and phentermine, and psychostimulant drugs when included, and their metabolites in the blood. Thus, the individual receives the combination therapy containing the specific ratio of each compound to achieve the desired effect. The combination therapy can also be in separate pharmaceutical carriers such as conventional dosage forms that are administered simultaneously such that each compound is administered at the specific ratio. For example, a first compound of the composition may be administered orally while the other compound of the composition may be administered by a non-oral route. The amounts of each compound are administered at a dosage of each to arrive at the specific ratio desired to achieve the desired effect.

### Compositions

The present disclosure is further directed to a composition including bupropion, phentermine and a psychostimulant drug. The composition is useful for treating attention deficit hyperactivity disorder in an individual in need. Through combination, reduction of adverse drug reaction and potentiation of the anti-psychiatric activity are intended by the combination of bupropion, phentermine and psychostimulant drug that can have different mechanisms of action.

The composition includes bupropion. As described herein, bupropion ((±)-2-(tert-Butylamino)-1-(3-chlorophenyl)propan-1-one) is an antidepressant that functions as a mild dopamine reuptake inhibitor and weak norepinephrine reuptake inhibitor. Bupropion also functions as a nicotinic acetylcholine receptor antagonist. Bupropion has been reported for use as a treatment for ADHD in minors and adults. The American Academy of Child and Adolescent Psychiatry noted that the evidence for treating ADHD with bupropion was weaker than for FDA-approved treatments.

The composition also includes phentermine (2-methyl-1-phenylpropan-2-amine). As described herein, phentermine acts on the hypothalamus portion of the brain to stimulate the adrenal glands to release norepinephrine. Phentermine can also stimulate the release of epinephrine or adrenaline.

The composition further includes a psychostimulant drug. Suitable psychostimulant drugs can be, for example, amphetamines, methylphenidate and combinations thereof.

As described herein, amphetamines stimulate the central nervous system and are used as single agents to treat ADHD and narcolepsy. Suitable amphetamines can be, for example, amphetamine mixed salts (e.g., ADDERALL®), dextroamphetamine ((RS)-1-phenyl-2-aminopropane)), levo amphetamine ((RS)-1-phenylpropan-2-amine) and lisdexamfetamine. The amphetamine mixed salts can be, for example, amphetamine salt and dextroamphetamine salt.

As described herein, methylphenidate (methyl phenyl(piperidin-2-yl)acetate) is a psychostimulant drug approved by the FDA for the treatment of ADHD. Administration of methylphenidate as a single agent for the treatment of ADHD can vary from about 10 mg to about 60 mg per day. Methylphenidate can result in increasing or maintaining alertness, combating fatigue and improving attention. Methylphenidate inhibits dopamine reuptake and also inhibits the reuptake of norepinephrine.

In contrast to the administration of bupropion, phentermine and psychostimulant drugs as single agents for treating ADHD, the combination therapy of the present disclosure allows for lower initial dosages of the compounds of the combination therapy than their dosages when administered as single agents. The lower initial dosages can contribute to a reduction in acquired tolerance. The lower dosages can also contribute to reducing the likelihood of side-effects caused by higher dosages. Additionally, the combination therapy allows for the administration over a prolonged period of time with less risk of dependence. Over time and/or if tolerance develops, the dosages can be increased for the continued treatment of ADHD. Thus, the lower initial dosages the compounds in the combination therapy can allow for a longer treatment period than when administered as single agents.

In one embodiment, the present disclosure is directed to a composition including a therapeutically effective amount of bupropion, a therapeutically effective amount of phentermine and a therapeutically effective amount of a psychostimulant drug. As used herein, “therapeutically effective amount” refers to that amount that provides a therapeutic effect for a given condition and administration regimen. It can be understood, however, that the total daily usage of the compounds and compositions of the disclosure can be decided by the attending physician within the scope of sound medical judgment. The specific therapeutically effective dose level for any particular patient can depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. Actual dosage levels of active ingredients in the pharmaceutical compositions disclosed herein can be varied so as to obtain an amount of the active compound(s) that is effective to achieve the desired therapeutic response for a particular individual, composition and mode of administration. The selected dosage level can depend upon the activity of the particular compound, the route of administration, the severity of the condition being treated and the condition and prior medical history of the individual being treated. However, it is within the skill of the art to start doses of the compound at levels lower than required to achieve the desired therapeutic effect and to gradually increase the dosage until the desired effect is achieved. Upon achieving the desired therapeutic effect, a fixed ratio of each agent can be determined to form a concentrated dosage of the combination therapy to accommodate a volume of fluid such that the combination therapy can be administered over a period of time such as, for example, a 24 hour period.

The active compounds (e.g., bupropion and phentermine, and psychostimulant drugs) can be administered in a convenient manner such as by oral administration, injection (subcutaneous, intravenous, etc.), inhalation, transdermal application, or rectal administration. Depending on the route of administration, the active compound may be coated in a material to protect the compound from the action of enzymes, acids and other natural conditions which may inactivate the compound. A preferred route of administration is by oral administration.

As used herein, “dosage” refers to physically discrete units suited as unitary dosages for the mammalian individual to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. Particularly suitable dosages of bupropion, phentermine and psychostimulant drugs are described herein.

The composition can also include a pharmaceutically acceptable carrier. The term “pharmaceutically acceptable carrier” refers to any suitable adjuvant, carrier, excipient, additive, antioxidant, osmolality adjusting agent, buffer, pH adjusting agent, or stabilizer. The pharmaceutically acceptable carrier may be in solid or liquid form such as, tablets, capsules, powders, solutions, suspensions, or emulsions. Particularly suitable pharmaceutically carriers may be, for example, saline (solution of 0.90% w/v of NaCl), half-normal saline (solution of 0.45% NaCl), quarter-normal saline (solution of 0.22% NaCl), D5W (a 5% dextrose in water solution), D5NS (a 5% dextrose in normal saline solution), Ringer's lactate, and other solutions known by those skilled in the art.

In view of the above, it will be seen that the several advantages of the disclosure are achieved and other advantageous results attained. As various changes could be made in the above methods without departing from the scope of the disclosure, it is intended that all matter contained in the above description shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A method for treating attention deficit hyperactivity disorder in an individual in need, the method comprising: administering to the individual a combination therapy that comprises bupropion and phentermine.

2. The method of claim 1, further comprising administering a psychostimulant drug.

3. The method of claim 2, wherein the psychostimulant drug is selected from the group consisting of an amphetamine, methylphenidate and combinations thereof.

4. The method of claim 3, wherein the amphetamine is selected from the group consisting of amphetamine mixed salts, dextroamphetamine, levoamphetamine, lisdexamfetamine and combinations thereof.

5. The method of claim 4, wherein the amphetamine mixed salts comprises amphetamine salt and dextroamphetamine salt.

6. The method of claim 1, wherein the administering is selected from the group consisting of orally, topically, transdermally, parenterally, subcutaneously, intravenously, intramuscularly, intraperitoneally, by intranasal instillation, by intracavitary or intravesical instillation, intraocularly, intraarterially, intralesionally, application to mucous membranes and combinations thereof.

7. The method of claim 1, wherein the buproprion comprises from about 75 mg to about 400 mg.

8. The method of claim 1, wherein the phentermine comprises from about 8 mg to about 37.5 mg.

9. A composition for treating attention deficit hyperactivity disorder comprising bupropion; phentermine; and a psychostimulant drug.

10. The composition of claim 9, wherein the psychostimulant drug is selected from the group consisting of an amphetamine, methylphenidate and combinations thereof.

11. The composition of claim 10, wherein the amphetamine is selected from the group consisting of amphetamine mixed salts, dextroamphetamine, and lisdexamfetamine.

12. The composition of claim 11, wherein the amphetamine mixed salts comprises amphetamine salt and dextroamphetamine salt.

13. The composition of claim 9, wherein the buproprion comprises from about 75 mg to about 400 mg.

14. The composition of claim 9, wherein the phentermine comprises from about 8 mg to about 37.5 mg.

15. The composition of claim 9, further comprising a pharmaceutically acceptable carrier.
